(19) United States
(12) Patent Application Publication
(10) Pub. No.: US 2007/0060857 A1
(43) Pub. Date: Mar. 15, 2007

(54) Knee brace

(76) Inventor: Anthony S. Testa JR., Henderson, NV (US)

Correspondence Address: GREENBERG TRAURIG, 3773 HOWARD HUGHES PARKWAY, SUITE 500 NORTH, LAS VEGAS, NV, 89169, US

(21) Appl. No.: 11/224,854

(22) Filed: Sep. 12, 2005

(51) Int. Cl. A61F 13/06; A61F 13/00

(52) U.S. Cl. 602/63; 602/62

(57) ABSTRACT

A tube-shaped knee brace having a first opening designed to be positioned above the wearer's knee joint, a second opening designed to be positioned below the wearer's knee joint, and a center opening disposed between the first and second openings, the center opening formed to accommodate the knee joint of the wearer wherein the brace has at least two pouches, each one of which is disposed along a length of the brace and wherein each one of the pouches contain a cylindrical spring generally formed in the shape of a helix configuration with advantages over the prior art.

### FIELD OF THE INVENTION

The embodiments of the present invention relate to a knee brace adapted to absorb impact and support a knee joint thus preventing injury to the Anterior Cruciate Ligament and Medial Collateral Ligament that surround the knee.

### BACKGROUND

Knee injuries, and in particular, injuries to the Anterior Cruciate Ligament (“ACL”) and/or the Medial Collateral Ligament (“MCL”), are common in activities of daily living and as a result of participation in recreational and professional sports. Health care professionals providing treatment for such injuries, frequently recommend patients use a knee support or brace. Additionally, medical professionals frequently use knee braces to prevent or to reduce the chance or severity of an initial injury. Normal range of motion for a healthy knee joint is from about 0 degrees to about 100 degrees. This normal range of motion may decrease however, as a result of knee pathologies. Current knee braces provide stability by incorporating stiffening means such as rods, stays or flattened interwoven helical springs. The physical characteristics and limitations of the stiffening means used in current devices prevent them from absorbing the energy of impacts while further restricting the knee range of motion of the wearer. Such reductions in the range of knee motion further restrict activities of daily living and reduce performance in sports.

There is a need for a knee brace that prevents injury by absorbing impact to the ACL and/or the MCL and does not further restrict the wearer's range of knee motion.

### SUMMARY

The embodiments in accordance with the present invention pertain to a flexible knee brace worn by wearer's during the course of performing activities of daily living and/or in the course of sporting activities so that lateral energy forces applied to the knee joint and in particular to the ACL and/or the MCL are absorbed by the device while not further restricting the wearer's range of motion of the knee joint. An embodiment of the impact absorption device in accordance with the present includes an elasticized knee brace generally formed to surround a knee wherein the brace comprises at least two pouches, each one of the pouches being positioned on opposite sides of the brace and wherein each one of the pouches contains a flexible helical spring formed in the shape of a circle. This configuration allows the spring to flex in both lateral and vertical directions to absorb impact to the ACL and/or the MCL thus reducing the chance of injury to those ligaments. To accommodate cylindrical springs of various dimensions, each one of the pouches has a resealable closure at one end of the pouch.

### BRIEF DESCRIPTION OF THE DRAWINGS

The present invention is described with respect to particular exemplary embodiments thereof and reverence is accordingly made to the drawings (which are not necessarily drawn to scale) in which:

FIG. 1 shows a front view of an embodiment of the present invention on a users lower leg;

FIG. 2 shows a side view of an embodiment of the present invention on a users lower leg;

FIG. 3 shows a side view of an embodiment of the present invention;

FIG. 4 shows a side view of a cylindrical spring of an embodiment of the present invention with a vertical and a lateral force applied thereto;

FIG. 5 shows a front view of a cylindrical spring of an embodiment of the present invention with lateral force applied thereto;

FIG. 6 shows a front view of an embodiment of the present invention; and

FIG. 7 shows a side view of an embodiment of the present invention.

### DETAILED DESCRIPTION

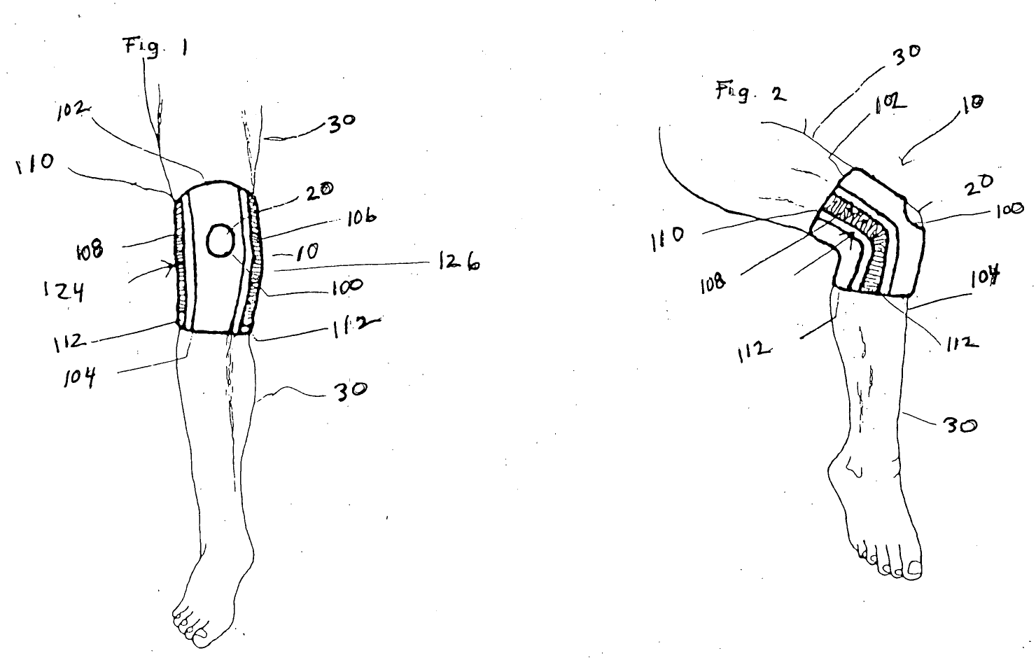

Referring now to the drawings, FIGS. 1, 2 & 3 illustrate embodiments of the present invention in which a brace 10 is positioned about a knee joint 20 of a lower leg 30 of a wearer. Brace 10 is generally tube shaped and formed to be generally, positionable on a wearer's knee 20 and adjacent leg areas above the knee joint 20 and below the knee joint 20. Tube shaped brace 10 has a center opening 100 formed to accommodate the knee joint 30 of the wearer and a first opening 102 designed to be positioned above the wearer's knee joint 20, a second opening 104 designed to be positioned below the wearer's knee joint 20.

Pouches 106 and 108 are disposed along generally opposite sides of brace 10. A first pouch 106 is disposed along a medial side 126 of a length of tube shaped brace 10 and a second pouch 108 is disposed along a lateral side 124 of a length of tube shaped brace 10 generally opposite first pouch 106. Each one of the pouches 106 & 108 has a first end 110 and a second end 112 comprising a length that begins at a point above the center opening 100 and terminates at a point below the center opening 100. Each one of the pouches 106 & 108 is formed to contain a cylindrical spring 114, best seen in FIGS. 4 & 5. Generally, each one of the pouches 106, 108 has a length from about 8 inches to about 12 inches.

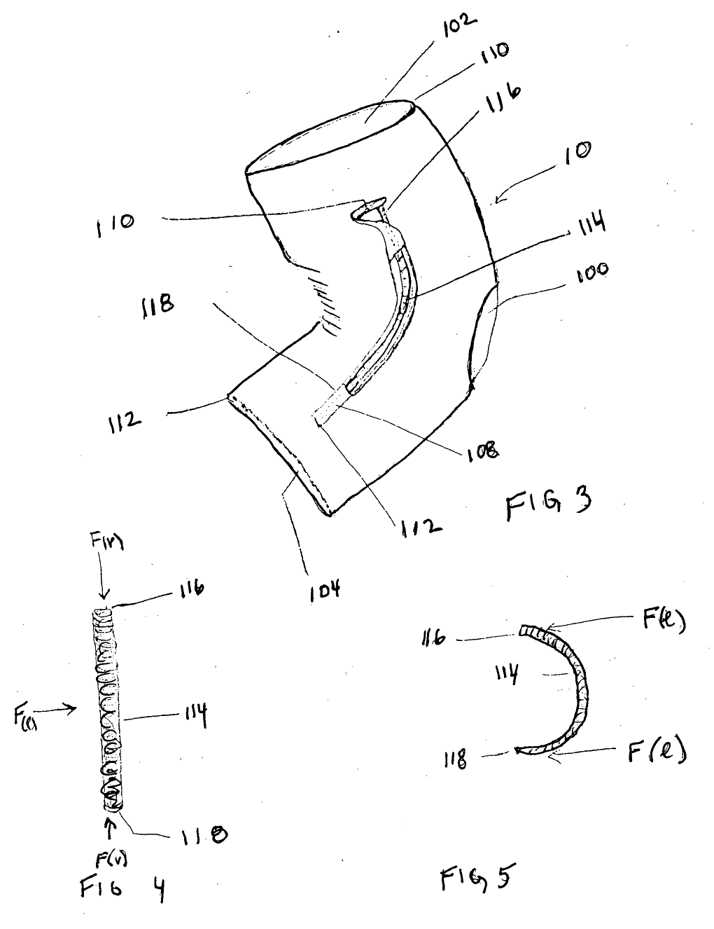

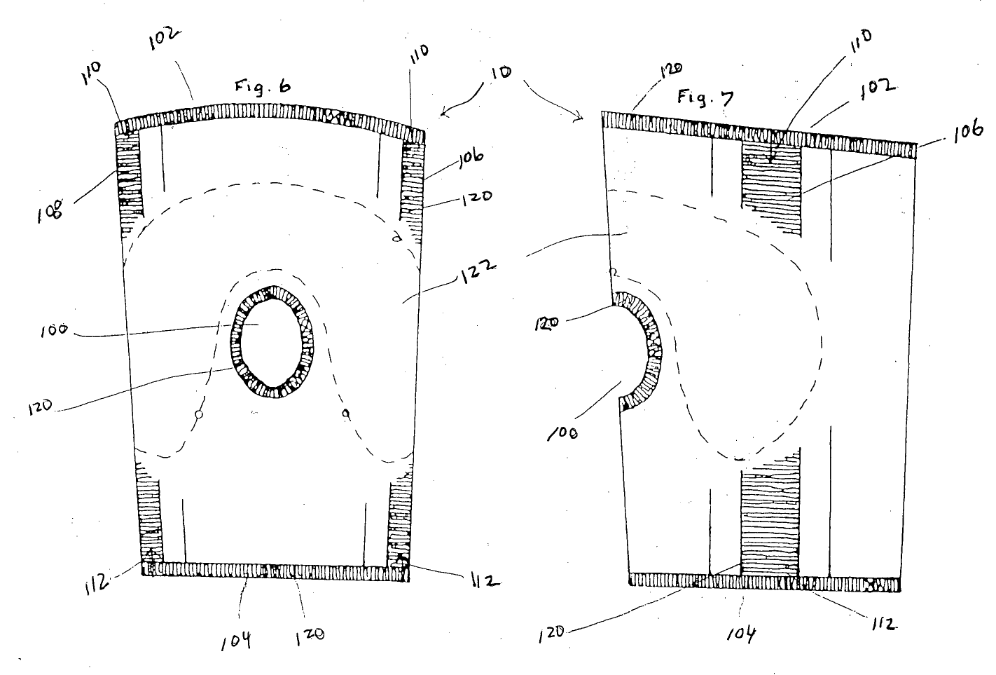

Generally formed in the shape of a helix, cylindrical spring 114 has a first end 116 and a second end 118 wherein the distance between the first end 116 and the second end 118 is approximately equal to a length of one of the pouches 106 or 108 respectively. Cylindrical spring 114 is of conventional construction and fabricated from wire curved into a shape it would assume if wound in single layer round a cylinder or cone. Suitable materials for cylindrical spring 114 include stainless steel or metal alloys having a diameter of approximately 0.054 millimeters. Those skilled in the art will recognize however, that other suitable materials and diameters may also be used. Preferably, cylindrical spring 114 is constructed of a metal alloy and has an external diameter from about ⅝ inch to about 1 inch, and more preferably about 9/16 inch. Cylindrical spring 114 has a length from about 8 inches to about 12 inches corresponding to the length of the pouches 106, 108. The external diameter of cylindrical spring 114 and the length of spring 114 may vary without departing from the spirit and scope of the invention. Cylindrical spring 114 is sufficiently flexible to allow first end 116 and second end 118 to move towards each other when an external lateral force (F1) is simultaneously applied to a position near the first end 114 and the second end 116, respectively. In some embodiments, cylindrical spring 114 is sufficiently flexible such that in response to an external lateral force (F1), first end 116 of cylindrical spring 114 tends towards the second end 118 of cylindrical spring 14, best shown in FIG. 5. When such lateral force (F1) is applied however to stiffing means such as spring steel members and/or flattened helical springs of the prior art, the stiffening means tend to deform and thereby restrict range of motion. In contrast, the helical springs 114 having a circular shape of the present invention, provide resistance to lateral forces which may impact a lateral side 124 or a medial side 126 of lower leg 30, best shown in FIG. 1, such lateral forces (F1) are characteristic of some activities of daily living and some sports. Embodiments of the present invention employing cylindrical springs having a resistance to vertical compression e.g., forces directed along a center axis of the spring, best shown in FIG. 4, provide weight-bearing support for wearer's of the knee brace 10 of the present invention.

In some embodiments, the first end 110 of each one of the pouches 106 & 108 comprises a means for opening the first end 110 to facilitate removal and replacement of cylindrical spring 114 from each one of the pouches 106 & 108. In this manner, cylindrical springs 114 having varying flexibility, length and diameter may be inserted into first end 110 of pouches 106 & 108. In some embodiments, the means for opening is fabricated from VELCRO®, but any button, snap, hook and eye or similar devices suitable for removably attaching fabrics together may be used.

Turning now to FIGS. 6 & 7, in some embodiments, tube shaped brace 10 is constructed of one or more pieces of woven elastic fabric stretchable in the circumferential direction relative the circumference of the lower leg 30 and has sufficient elasticity to generally form to the shape of the wearer's knee 20 and lower leg 30. In some embodiments, a layer of latex rubber sheeting or other suitable material, not shown, is connected with the woven elastic fabric of brace 20 by a tight surge stitch 120 to reinforce the woven fabric of brace 10. Other conventional methods of fabrication may be used. In some embodiments, the rubber sheeting has a thickness of approximately 10 millimeters. Center opening 100 is reinforced by surge stitch 120. In some embodiments, brace 10 comprises a protective pad 122 of conventional construction fabricated within layers of the woven fabric of brace 10. Each one of the pouches 106 & 108 is formed of fabrics having sufficient elasticity to contain the movement of spring 114 within pouch 106 and/or 108, respectively. In some embodiments, pouches 106, 108 are formed of a cotton web jute material and stitched together during fabrication with tube shaped brace 10. Materials and construction of brace 10, pouches 106 & 108, protective pad 122 and stitching 120 may vary without detracting from the scope of the present invention.

A method for absorbing impact to the ACL and/or the MCL without further restricting the range of motion of the wearer of a knee brace of the present invention comprises the following steps: providing an elastic knee brace having at least two pouches disposed along a length of the knee brace wherein the pouch is formed to contain a cylindrical spring generally formed in the shape of a helix; inserting at least one of a helix; inserting at least one cylindrical spring into at least one of the pouches of the knee brace whereby the wearer's range of motion is not restricted.

It should be understood that the particular embodiments above are only illustrative of the principles of the present invention and various modifications could be made by those skilled in the art without departing from the scope and spirit of the invention thus, the scope of the present invention is limited only by the claims that follow.

I/We claim:

1. A protective brace for a knee joint comprising:
a tube-shaped brace formed of an elastic material, the tube-shaped brace formed to be generally positionable on a wearer's knee and adjacent leg areas above a knee joint and below the knee joint, the tube-shaped brace having a first opening designed to be positioned above the wearer's knee joint, a second opening designed to be positioned below the wearer's knee joint, and a center opening disposed between the first and second openings, the center opening formed to accommodate the knee joint of the wearer;
at least two cylindrical pouches, each one of said pouches formed of an elastic material, a first one of said pouches being disposed along a length of the tube-shaped brace and a second one of said pouches disposed along a length of the tube-shaped brace generally opposite the first pouch, wherein a first end of each one of the pouches begins at a point above the center opening and a second end of each one of the pouches terminates at a point below the center opening, each one of the pouches formed to contain a cylindrical spring; and,
a first cylindrical spring retained in the first pouch and a second cylindrical spring retained in the second pouch, each spring having a first end and a second end, wherein a distance between the first and second ends is approximately equal to a length of one of the pouches.

2. The brace of claim 1 wherein, each one of said cylindrical springs has a length of from about 8 inches to about 12 inches.

3. The brace of claim 1 wherein, each one of said cylindrical springs has an external diameter from about ⅝ inch to about 1 inch.

4. The brace of claim 1 wherein the pouches are formed of one or more elastic fabrics, the elastic being attached to the brace.

5. The brace of claim 1 wherein an end of each one of the pouches comprises an opening means for removal of the cylindrical spring retained within each one of the pouches.

6. A protective brace for a knee joint comprising:
a tube-shaped brace formed of an elastic material, the tube-shaped brace formed to be generally positionable on a wearer's knee and adjacent leg areas above a knee joint and below the knee joint, the tube-shaped brace having a first opening designed to be positioned above the wearer's knee joint, a second opening designed to be positioned below the wearer's knee joint, and a center opening disposed between the first and second openings, the center opening formed to accommodate the knee joint of the wearer;
at least two cylindrical pouches, each of said pouches formed of an elastic material, a first one of said pouches being disposed along a length of the tube-shaped brace medial to the wearer's knee and a second one of said pouches disposed along a length of the tube-shaped brace lateral to the wearer's knee wherein a first end of each one of the pouches begins at a point above the center opening and a second end of each of the pouches terminates at a point below the center opening, each of the pouches formed to retain a cylindrical spring; and
a first cylindrical spring retained in the first pouch and a second cylindrical spring retained in the second pouch, each cylindrical spring having a first end and a second end, wherein, each of the cylindrical springs has an external diameter from about ⅝ inch to about 1 inch and wherein each of the cylindrical springs has a length of from about 8 inches to about 12 inches.

7. A method for absorbing impact to the Anterior Cruciate Ligament and/or the Medial Collateral Ligament without restricting the range of motion of the wearer of a knee brace of the present invention comprising the following steps:
providing an elastic knee brace having at least two pouches disposed along a length of the knee brace wherein each pouch is formed to contain a cylindrical spring generally formed in the shape of a helix; and
inserting at least one cylindrical spring generally formed in the shape of a helix into at least one of the pouches of the knee brace whereby, the range of motion of the knee of the wearer is not restricted.

8. A method for absorbing impact to a ACL and/or a MCL comprising:
positioning an elastic knee brace on a wearer such that the knee brace generally surrounds the wearer's knee except for an opening for accommodating a wearer's knee cap, and
wherein the elastic knee brace includes two or more pouches disposed along a length of the knee brace wherein each pouch retains a cylindrical spring, the pouches disposed along opposite sides, namely a medial and lateral portion, of the knee brace such that the cylindrical springs absorb lateral impact to the wearer's knee.
